(19) United States
(12) Patent Application Publication
(10) Pub. No.: US 2009/0281627 A1
(43) Pub. Date: Nov. 12, 2009

(54) Filling material for filling a vertebral body cavity, intervertebral prosthetic disc nucleus and vertebroplasty prosthesis comprising such a material

(75) Inventor: Dominique Petit, Verton (FR)

Correspondence Address: HARNESS, DICKEY & PIERCE, P.L.C., P.O. BOX 828, BLOOMFIELD HILLS, MI, 48303, US

(73) Assignee: Spinevision, Paris (FR)

(21) Appl. No.: 11/921,335

(22) PCT Filed: Jun. 2, 2006
(86) PCT No.: PCT/FR2006/001261

§ 371 Date: Apr. 9, 2009

(30) Foreign Application Priority Data
Jun. 2, 2005 (FR) 0505594

(51) Int. Cl. A61F 2/44; A61F 2/02

(52) U.S. Cl. 623/17.12; 623/23.72

(57) ABSTRACT

The invention concerns in particular an invertebral prosthetic disc nucleus (1) in the form of a flexible filament (5) of a biocompatible elastomer. The filling material is adapted for injecting by means of a hollow needle (4) into the cavity (2) of the annulus. The filament winds when contacted with the walls (6) and with its own segments so as to form an entanglement filling the cavity.

The present invention relates to a filler material intended to fill a cavity in the body of a vertebra.

The present invention also relates to a prosthesis of a nucleus of an inter-vertebral disc and a vertebroplasty prosthesis comprising such a filler material.

An inter-vertebral disc consists of an annulus fibrosis which gives the disc its strength and a nucleus in pasty/gelatinous form.

Some pathologies require surgery at the level of the disc. This is for instance the case of the disc herniation which is a leakage of the nucleus through a hole, occurring in the annulus. This can also be the case with sagging of the disc height which comes with age

In the state of the art, inter-vertebral prostheses are known which are intended to replace discs. Such prostheses make it possible for two vertebras to go on moving with respect to one another, and in a controlled way after the ablation of the disc.

However, surgery aims at totally replacing a disc by prosthesis, and it is a rather severe and invasive surgery. Besides, this type of surgery can be made only once through the same channel, first because of adhering tissues.

Now, in many cases, it is not useful to replace the whole disc but simply the nucleus. Besides, pathologies like sagging of disc height can be treated by adding some nucleus material.

A new technology known as IDET (Intradiscal Electrothermal Annuloplasty) consists in inserting a hollow needle through the annulus and have a heating wire called electrothermal catheter go through this needle, so that the catheter encompasses the external periphery of the nucleus.

Such a technology opened the way to the development of nucleus replacement methods. Such methods use nucleus prostheses which can be introduced using a hollow needle going through the annulus.

A first prosthesis known is described in U.S. Pat. No. 6,533,817. It concerns a hydrogel polymer pad introduced in the dehydrated state. Once the pad is introduced, it is hydrated so as to fill the inside of the annulus. This prosthesis includes many disadvantages. First, the hole required for introducing the pad is of a relatively large size. Then, the pad does not completely fill the inside of the annulus and consequently tends to move and does not correctly fulfill its function as a dumper. Besides the hydrogel polymer is not bio-stable and may deteriorate rather quickly.

A second prosthesis known is obtained by introducing a polymer in the liquid form through a small hole bored in the annulus, the polymerization occurring inside the annulus. Such prosthesis also entails a certain number of problems. First, the polymerization must occur at sufficiently high temperatures, so that polymers do not become toxic during the polymerization, generally above 45°. But, this temperature is close to the maximum temperature acceptable by the body. An important risk exists of destroying cells and more particularly those of the annulus during the polymerization operation.

As the polymer is introduced in the liquid form, risks of liquid leakage exist, if the annulus is bored with another hole than the introduction hole, with neurological risks which are very severe.

As for the vertebroplasty, the surgical operation consists in injecting certain orthopedic cement into a vertebral body. Vertebroplasty more particularly aims at stabilizing vertebral bodies. However, the vertebroplasty has a certain number of disadvantages, such as for instance cement leakage.

The present invention provides nucleus and vertebroplasty prosthesis which do not have the disadvantages of the prosthesis known in the state of the art.

For this purpose and according to a first aspect, the invention relates to a filler material intended to fill a cavity of the vertebral body, characterized in that it is in the form of a flexible filament, said material being adapted for injection into the conduit of a hollow needle, said material having at a temperature under 45° C., a flexibility which makes it possible for winding in said cavities so as to form an entanglement which fills such cavity.

According to a second aspect, the invention relates to a nucleus prosthesis intended to fill the annulus of an inter-vertebral disc comprising such a filler material. In this application, the filler material is adapted to the injection in a hollow needle going through the annulus wall.

According to a third aspect of the invention, the invention relates to a vertebroplasty prosthesis intended to fill a bone cavity and comprising such a filler material. In this application, said material is adapted to the injection into the conduit of a hollow needle going through the bone.

Whatever the application, the filler material is injected into the conduit of the needle and it penetrates into the cavity in the form of a flexible filament, cavity in which it advances while bending when coming in contact with the walls and other parts of itself until it forms an entanglement which totally fills such cavity.

Advantageously, a filler material can be injected so as to form a substantially ordered entanglement such as in the form of a filament ball or reel, or a disordered entanglement.

Advantageously, the filler material is in the form of a flexible filament, the section of which is adapted to the injection into the conduit of a needle.

The filament injection device can advantageously be a glue gun type.

According to another aspect of the present invention, said filler material has a flexibility during injection which is higher than its flexibility at rest, so that it is flexible enough during the injection to completely fill the inside of the cavity and rigid enough to guarantee, once injected, the stable reinforcement. In the particular case of the nucleus prosthesis, the filler material must have a sufficient rigidity to ensure the preservation of the inter-vertebral height.

Advantageously, said filler material has said rigidity at rest at the human body's temperature and said injection flexibility at the injection temperature which is higher than the human body's temperature.

The filler material is heated up to the injection temperature prior to being injected, so that it gets the flexibility required for the filling operation. Once injected, the material goes down to the human body's temperature, which gives it the rigidity at rest.

Advantageously, the injection device of the glue gun type is provided with an adjusted heating device so that the filament emitted through the outlet nozzle is at the injection temperature.

Advantageously, said injection temperature is lower than 45° C.

Injection temperature means the temperature of the filler material when it goes through the annulus holes or the bone. The heating temperature for example at the nozzle of an injection device can be higher than 45° C.

Advantageously, said material is a biocompatible elastomer.

Advantageously, said material is polycarbonate-urethane.

In addition to being a biocompatible elastomer, the polycarbonate-urethane is bio-stable which means that it is not bio-resorbable, nor bio-degradable.

The present invention will be better understood when reading the description of a non-limitative exemplary embodiment which follows, while referring to the appended drawings in which:

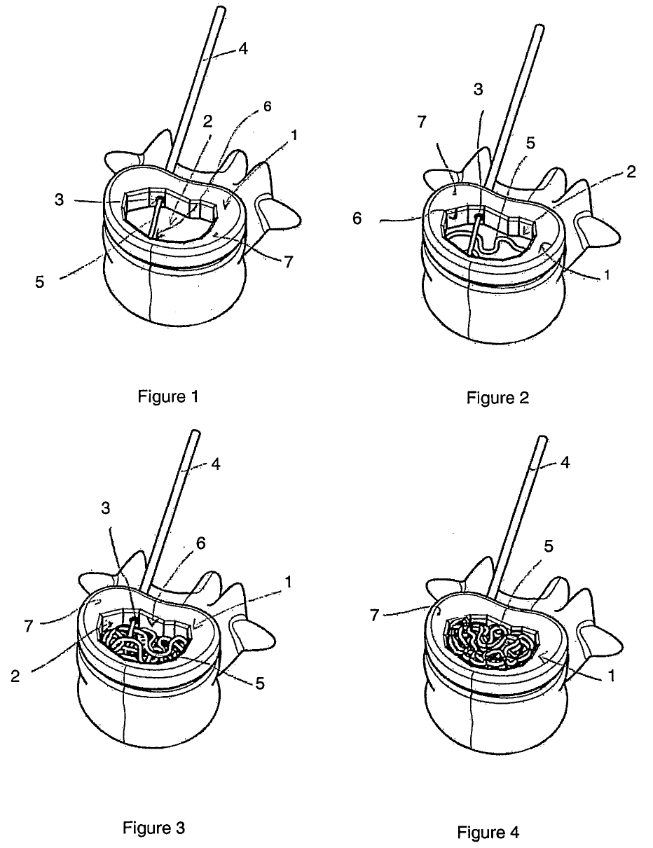

FIGS. 1 to 4 show schematic views of an inter-vertebral disc on which a nucleus prosthesis is fitted according to the present invention, at various stages of the fitting of such prosthesis.

A nucleus prosthesis according to the present invention is in the form of a flexible filament of a biocompatible elastomer like polycarbonate-urethane.

Such a filament can be injected into the inner cavity of the annulus using a hollow needle which goes through the annulus wall. The injection is controlled by an injection device of the glue gun type.

The filament is obtained from a material in the form, at the injection device inlet, of a filament, preferably a substantially straight filament, a viscous paste and/or a powder.

When the material to be injected is in the form of a filament, said filament can have either the diameter expected for its injection in the needle, or a greater diameter, an end nozzle of the injection device giving it the expected diameter then. Advantageously, the filament is positioned in bulk or wound in the injection device so as to form for example a filament reel.

According to a particular configuration, the filler material can be composed of several components in the form of a fluid, a viscous paste and/or a powder. Then, it will be advantageous to provide an injection device comprising as many containers as components to be mixed, the mixture of the components with a view to obtaining the filler material to be inject being carried out in a common chamber of the injection device.

According to an advantageous embodiment of the present invention, the end nozzle of injection device is associated with a heating device so that at the time of going through the annulus walls, the material is at a temperature comprised between the human body's temperature and 45° C.

FIGS. 1 to 4 show various stages of the injection operation of a prosthesis according to the invention, the filler material already being, prior to the injection, in the form of a filament.

The filament 5 of the filler material goes into the conduit of the hollow needle 4, which goes through the annulus 7 wall of the disc 1 through a hole 3. It penetrates into the inner cavity 2 in which it advances while bending when coming into contact with walls 6 and other segments of its own body, until it forms an entanglement which completely fills the cavity.

1. A filler material intended to fill a cavity in a vertebral body comprising a flexible filament, said material being adapted to the injection into the conduit of a hollow needle, said material having, at a temperature under 45° C., a flexibility making it possible for the filament to wind in said cavity so as to form an entanglement filling such cavity.

2. A filler material according to claim 1, having a flexibility, during the injection, which is higher than its flexibility at rest, so that it is flexible enough during the injection to completely fill the cavity and rigid enough to secure once injected a stable support.

3. A filler material according to claim 2, having said rigidity at rest at the human body's temperature and said injection flexibility at an injection temperature higher than the human body's temperature.

4. A filler material according to claim 3, wherein said injection temperature is lower than 45° C.

5. A filler material according to claim 1, having the form of a flexible filament having a section adapted to the injection into the conduit of the needle.

6. A filler material according to claim 1, wherein said material is a biocompatible elastomer.

7. A filler material according to claim 6, wherein said material is polycarbonate-urethane.

8. A nucleus prosthesis intended to fill the annulus of an inter-vertebral disc comprising a filler material according to claim 1, said material being adapted to the injection be injected into a conduit of a hollow needle going through the annulus wall, and having, at a temperature lower than 45° C., a flexibility which makes it possible for the filament to wind in the inner cavity of the annulus, so as to form an entanglement filling such cavity.

9. A prosthesis according to claim 8, wherein said filler material has a flexibility during injection which is higher than its flexibility at rest, so that it is flexible enough during the injection to completely fill the inner cavity of the annulus and rigid enough to guarantee, once injected, the preservation of the inter-vertebral height.

10. A vertebroplasty prosthesis intended to fill a bone cavity comprising a filler material according to claim 1, said material being adapted to the injection into the conduit of a hollow needle going through the bone, said material having, at a temperature lower than 45° C., a flexibility making it possible for the filament to wind in the inner cavity of the bone, so as to form an entanglement filling such cavity.
